(19) United States
(12) Patent Application Publication
(10) Pub. No.: US 2010/0130838 A1
(43) Pub. Date: May 27, 2010

(54) Infrared Temperature Measurement of Strip

(76) Inventors: Mahyar Z. Kermani, Pleasanton, CA (US); Sharbel Noujaim, Menlo Park, CA (US); Thomas Jetter, Mainz, DE (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/603,204

(22) Filed: Oct. 21, 2009

Related U.S. Application Data
(60) Provisional application No. 61/107,002, filed on Oct. 21, 2008.

(51) Int. Cl. A61B 5/1455; A61B 5/1468

(52) U.S. Cl. 600/310; 600/347

(57) ABSTRACT

Provided are systems and methods that permit the direct assessment of temperature on an electrochemical test strip, including at the reaction site of the strip, through the inclusion of an infrared sensor within the biosensing instrument. Analyte measurement systems are provided in which an infrared sensor is used to assess temperature associated with a test strip, and the acquired temperature data is used to modulate data regarding an analyte in a biological sample, thereby providing a more accurate measurement of the analyte.

### CROSS REFERENCE TO RELATED APPLICATION

The present application claims priority to U.S. Provisional App. No. 61/107,002, filed Oct. 21, 2008, which is hereby incorporated by reference in its entirety.

### TECHNICAL FIELD

The present invention relates to the detection of analyte levels by medical diagnostic systems such as blood glucose meters.

### BACKGROUND

Biosensing instruments are used for the detection of various analytes (e.g., glucose and cholesterol) in blood samples. For example, blood glucose meters are medical diagnostic instruments used to measure the level of glucose in a patient's blood, and may employ disposable sample strips having a well or reaction zone for receiving a blood sample. Some meters include sensor assemblies that determine glucose levels by measuring the amount of electricity that can pass through a sample of blood, while other meters include sensor assemblies that measure how much light reflects from a sample. A microprocessor of the meter then uses the measured electricity or light from the sensor assembly to compute the glucose level and displays the glucose level as a number.

An important limitation of electrochemical methods of measuring the concentration of a chemical in blood is the effect of confounding variables on the diffusion of analyte and the various active ingredients of the reagent. For example, analyte readings are influenced by the ambient temperature that surrounds the sample well or reaction zone. As with any electrochemical sensing method, transient changes in temperature during or between measurement cycles can alter background signal, reaction constants and/or diffusion coefficients. Accordingly, a temperature sensor may be used to monitor changes in temperature over time. A maximum temperature change over time threshold value can be used in a data screen to invalidate a measurement. Absolute temperature threshold criteria can also be employed, wherein detection of high and/or low temperature extremes can be used in a data screen to invalidate a measurement. The microprocessor of a glucose sensor can make a determination as to whether the temperature of the testing environment is within predetermined thresholds, and prohibit a user from running a test if accuracy would be negatively affected. It is important, therefore, that any temperature sensing elements of the glucose meter not be affected by heat generated within the glucose meter (e.g., by a backlight liquid crystal display).

The temperature sensing elements of the glucose meter should have access to the ambient temperature surrounding the meter. In view of the temperature sensitivity of the biochemical reactions that are interpreted by a biosensing device, ambient temperature values that are obtained by temperature sensors are directly used during the assessment of analyte levels in the sample. As a consequence, even relatively minor variations in sensed ambient temperatures can create fluctuations in biochemical readings and result in erroneous outputs. Because the outputs provided by the biosensing device is intended to influence the patient's decisions regarding, inter alia, dosing of medication, it is very important that erroneous readings be avoided. Thus, biosensing instruments should include means for avoiding erroneous outputs that result from inaccurate or misleading ambient temperature readings.

Various prior art instruments employ internal or external thermal sensors in order to acquire information about the ambient temperature (see e.g., U.S. Pat. No. 5,405,511; U.S. Pub. No. 2006/0229502), while other instruments attempt to control the temperature of the reaction zone, and still other devices attempt to obtain indirect measurements of blood sample temperature by use of complex algorithms that rely upon the use of an ambient temperature sensor in combination with AC admittance measurements (see U.S. Pat. No. 7,407,811).

While sensors that are sensitive to ambient temperature are capable of rapidly reacting to a temperature change and thereby provide timely information, under certain circumstances this attribute can have undesired consequences. For example, when a biosensing instrument that is normally held in a user's hand is placed on a tabletop, a rapid temperature change may occur that can bias subsequent biochemical readings until ambient temperature readings have stabilized. As for instruments that attempt to control the temperature of the reaction zone, if the biosensing instrument is battery-driven, it becomes impractical to control the reaction zone temperature as this requires too great a power drain from the instrument's battery. Furthermore, certain approaches, such as that described in U.S. Pat. No. 7,407,811 do not provide a universal solution to the problem of estimating ambient temperature; the approach described in that patent is designed for use with a specific glucose strip, and if the strip chemistry or strip geometry changes, the disclosed algorithm must be modified. There remains a need for temperature sensing systems that can overcome these problems and otherwise improve the accuracy of analyte measurements by biosensing instruments.

### SUMMARY

In one aspect, the present invention is directed to methods comprising using an infrared sensor to assess temperature associated with a test strip that is inserted into an analyte measurement system, wherein the system comprises a housing; an analyte measurement component disposed within the housing, or proximate the housing, and having an aperture for receiving the test strip, wherein the analyte measurement component measures an analyte on the test strip, thereby providing analyte measurement data; the infrared sensor disposed at least partially within the housing; and a processor disposed within the housing that uses temperature data from the infrared sensor to modulate the analyte measurement data.

In another aspect, the present invention provides systems comprising a housing; an analyte measurement component disposed within the housing, or proximate the housing, and having an aperture for receiving a test strip, wherein said analyte measurement component measures an analyte on the test strip, thereby providing analyte measurement data; an infrared sensor disposed at least partially within the housing; and a processor disposed within the housing that uses temperature data from the infrared sensor to modulate the analyte measurement data.

### BRIEF DESCRIPTION OF THE DRAWINGS

FIG. 1 depicts the results of an experiment designed to assess the infrared transmission of analyte test strips.

FIG. 2 provides the results of an experiment designed to assess the infrared reflectance of analyte test strips.

FIG. 3A depicts an exemplary embodiment featuring an infrared sensor disposed within the housing of an analyte measurement system that can measure a portion of a test strip that is inserted into the aperture of the analyte measurement component.

FIG. 3B provides the results of infrared temperature measurement of a portion of a test strip that is inserted into the aperture of the analyte measurement component.

FIG. 4 depicts a partially transparent side view of an exemplary analyte measurement system in accordance with the present methods and systems.

FIG. 5 depicts (A) an experimental system comprising an infrared sensor and light guide; (B) the results of the measurement of the temperature of a standard glucose strip positioned outside of the experimental device, and (C) the error observed with respect to the temperature measurement.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

While the measurement of ambient temperature surrounding a biosensing instrument by means of a sensor (e.g., a thermistor, thermometer, or thermocouple device) can provide information that can be used to improve the accuracy of measurement of one or more analytes in a biological sample, such temperature measurement represents an estimation of the actual temperature at the site of the relevant electrochemical reaction (often the well or reaction zone of a test strip). In addition, biosensing instruments are usually compact devices, and often incorporate liquid crystal displays with backlight, large processors for data processing, RF components for wireless communication, and many other electronic components or subassemblies; such components consume power and they result in heat dissipation. The interior temperatures of compact devices with internal power dissipation can rise significantly above the ambient temperature, which can mean that a measurement of temperature using an internal thermistor may not be representative of the actual ambient temperature. This can in turn influence analyte readings derived from a sample well or reaction zone of a test strip.

It has presently been discovered that a direct measurement of the temperature at the reaction site can greatly improve an instrument's ability to conduct accurate measurements of an analyte in the test sample by allowing the instrument to compensate for the actual temperature conditions affecting the reaction of the sample with the strip's sensor assembly. The present invention permits the direct assessment of temperature associated with an electrochemical test strip, including at the reaction site of the strip, through the inclusion of an infrared sensor as a component of the biosensing instrument. Direct measurement of temperature through the use of infrared radiation greatly improves the ability of the biosensing instrument to provide accurate readings regarding analyte levels, which has a positive effect on a user's ability to obtain the medical information required to make appropriate and timely decisions regarding medication, consultation with a doctor or nurse, or other treatment options. Furthermore, the present invention permits a temperature determination that is independent of the device orientation, power fluctuation, and other factors that can skew temperature readings in devices in which a single non-infrared sensor is used to estimate ambient temperature.

In one aspect, the present invention is directed to methods comprising using an infrared sensor to assess temperature associated with a test strip that is inserted into an analyte measurement system, wherein the system comprises a housing; an analyte measurement component disposed within the housing, or proximate the housing, and having an aperture for receiving the test strip, wherein the analyte measurement component measures an analyte on the test strip, thereby providing analyte measurement data; the infrared sensor disposed at least partially within the housing; and, a processor disposed within the housing that uses temperature data from the infrared sensor to modulate the analyte measurement data.

In another aspect, the present invention provides systems comprising a housing; an analyte measurement component disposed within the housing, or proximate the housing, and having an aperture for receiving a test strip, wherein said analyte measurement component measures an analyte on the test strip, thereby providing analyte measurement data; an infrared sensor disposed at least partially within the housing; and a processor disposed within the housing that uses temperature data from the infrared sensor to modulate the analyte measurement data.

Unless otherwise specified, the description of a particular embodiment, feature, component, or functionality applies both to present methods and the present systems. For example, reference to a “system” applies both to the “analyte measurement systems” of the present methods and to the “systems” as separately claimed.

The analyte measurement system may be a glucose or cholesterol monitor device. Such devices may include a port or other component that is used to accommodate a test strip that is inserted by the user either before or after the biological sample has been placed on an appropriate location on the strip. The test strip is preferably an electrochemical test strip, i.e., a strip that is configured for generating electrical signals that reflect the concentration of one or more analytes in a biological sample such as blood. The temperature that is “associated with a test strip” is preferably the temperature of the air immediately adjacent to the test strip (e.g., within about 5 mm or less, about 3 mm or less, or about 1 mm or less from a surface of the test strip), the temperature of one or more portions of the test strip itself, the temperature of the sample on the test strip, or any combination thereof, i.e., to a plurality of readings corresponding to any combination of the preceding temperatures. For example, where the test strip has a length l, the present methods and systems can be used to assess the temperature on a portion of the test strip that is located at a distance of no more than about ⅓l from the end of the test strip that is inserted into the aperture of the analyte measurement component of the analyte measurement system. In other embodiments, where the test strip has a length l, the temperature may be assessed on a portion of the test strip that is located at a distance that is greater than about ⅓l from the end of the test strip that is inserted into the aperture of the analyte measurement component. When the test strip has a length l, the temperature may also or alternatively be assessed on a portion of the test strip that is located at a distance that is greater than about ⅔l from the end of the test strip that is inserted into the aperture of said analyte measurement component.

The temperature that is associated with the test strip may be assessed more than one time. For example, the temperature may be assessed two or more times with respect to the temperature of the air immediately adjacent to the test strip (i.e., within about 5 mm or less, about 3 mm or less, or about 1 mm or less from a surface of the test strip), the temperature of one or more portions of the test strip itself, the temperature of the sample on the test strip, or any combination thereof. The same location on or near the test strip may be assessed more than one time, or each of two or more different locations may be assessed one or more times. Some or all of the data that is derived from the assessment of temperature associated with the test strip (i.e., some or all of the one or more assessed temperatures associated with the test strip) may be used to modulate the analyte measurement data that is measured by the analyte measurement component of the system. When multiple temperatures associated with the test strip are assessed, the individual assessments may occur at any desired interval over time; such intervals may be fractions of seconds, seconds, or minutes, and the intervals may be of the same duration or one or more different durations.

The present systems include a housing that substantially defines an internal space. The housing may be made from any suitable material and may adopt any appropriate configuration that can accommodate those components of the system that must be internal to the housing. Many biosensing instruments have housings that comprise a plastic shell assembled from one or more molded parts. For example, the housing may be a shell comprising a first and a second half, one half forming the “upper” portion of a device in a horizontal resting position (such as on a tabletop), and the other half forming the “lower” portion of the device, the two halves having been configured to allow their secure attachment to one another in order to form an integrated shell, and to accommodate internal components, components that may be partially external to the housing (such as switches, interface buttons, display components, etc.), features necessary for the assembly of the housing (such as interlocking parts, or screw or rivet holes), batteries (i.e., the housing may include a battery port and/or battery door), air vents, and the like. The housing may also feature one or more coated sections that enhance the user's ability to grip the biosensing instrument, such as rubber gripping portions on the outer lateral sides of the housing. Those skilled in the art will readily appreciate the size, shape, and material parameters that may suitably be used to form a housing of an analyte measurement system.

The analyte measurement component is disposed within the housing or proximate the housing. In other words, the analyte measurement component may be partially or completely disposed within the housing, may be mounted or otherwise affixed to the housing, may be at least partially defined by the housing, or may be any combination thereof. The analyte measurement component includes an aperture for receiving the test strip and can measure an analyte on the test strip, i.e., can measure an analyte that is present within a biological sample on the test strip, thereby providing analyte measurement data, which can be communicated to another component of the system. Analyte measurement components are found in traditional biosensing instruments, for example, whereby the aperture is located at one end of the housing (which may in fact be molded such as to define the aperture) and includes electrical components that contact the inserted end of a test strip and receive the electrical signals that have traveled to the inserted end of the test strip from the end of the strip that holds the biological sample. The aperture typically includes a groove or slot having the same width as a test strip, into which the test strip is inserted by the user. The electrical components interface with processing equipment inside the housing, such as a microprocessor, to which the electrical components supply analyte measurement data corresponding to the signals received from the test strip. Various configurations for the analyte measurement component will be readily appreciated by those having ordinary skill in the art, who will recognize that the analyte measurement component of the present invention may be configured in a manner that is similar to analyte measurement components of traditional biosensing instruments.

Pursuant to the present invention, the infrared sensor assesses a temperature associated with the test trip; it has been determined in the context of the present invention that the material used to construct electrochemical test strips is suitable for infrared measurement. The infrared sensor is disposed at least partially within the housing. In some embodiments, the infrared sensor may be attached to the outside of the housing. Preferably, the infrared sensor is disposed substantially within the housing; in other words, most or all of the infrared sensor is preferably disposed within the housing, although one or more components associated with the infrared sensor (such as one of those specified infra) may be at least partially disposed outside of the housing and/or extend through the housing from the internal space to the ambient environment outside of the housing. Infrared temperature sensors exist in a number of different configurations, but generally speaking, each uses a lens to focus infrared energy emitted from a target onto an internal detector, which converts the energy to an electrical signal which in turn can be converted into temperature data based on the sensor's calibration equation and the target's emissivity. Preferably, the infrared sensor should be sized so as to fit substantially within the housing.

Suitably configured infrared sensors are commercially available, for example, from Melexis Microelectronic Systems (Concord, N.H.), which sells an appropriately sized sensor that is said to have a temperature accuracy of ±0.5° C. over a wide temperature range (Cat. No. MLX90614), or Heimann Sensor GmbH (Dresden, Germany), which offers an “ultrasmall” thermopile sensor (Cat. No. HMSZ11). Other examples include the ZTP 135 series of infrared sensors from General Electric Sensing & Inspection Technologies (Billerica, Mass.), and the TPS series of sensors from PerkinElmer Optoelectronics (Fremont, Calif.). Additional parameters for the infrared sensor are discussed infra. Preferably, the infrared sensor has an accuracy of ±2° C. or better, an accuracy of ±1° C. or better, or an accuracy of ±0.5° C. or better. This accuracy should be maintained within a range of ambient temperatures in which it can be expected the user will attempt to operate the biosensing instrument, for example, within the range of 0° C. to 60° C., while the sensor temperature itself could vary between 0° C. to 50° C.

The infrared sensor may be positioned at a location substantially within the housing that is sufficiently distanced from a heat source (e.g., a liquid crystal display, a microprocessor, or any other source of heat within the biosensing device) such that it is unnecessary to provide physical insulation of the infrared sensor, which is heat-sensitive and self-calibrates according to the ambient temperature around the sensor. However, if the analyte measurement system is configured such that the infrared sensor is proximate to a heat source, it may be necessary to insulate the infrared sensor. Because the infrared sensor may include an embedded thermistor, the infrared sensor can measure the strip or ambient temperature accurately regardless of the temperature of the infrared sensor itself, and, consequently, there may be no need to isolate the infrared sensor completely from the heat source.

Typically, only the 0.7 to 14 micron band, inclusive, is used for infrared temperature measurement, and the infrared sensor according to the present invention may use any infrared wavelength within this range. In a preferred embodiment, the infrared sensor uses radiation having a wavelength of about 8 microns to about 14 microns to assess a temperature associated with the test strip. Where the infrared temperature sensor performs more than one temperature assessment associated with the test strip, each respective reading may use the same wavelength of infrared radiation, or may use different wavelengths within the prescribed range.

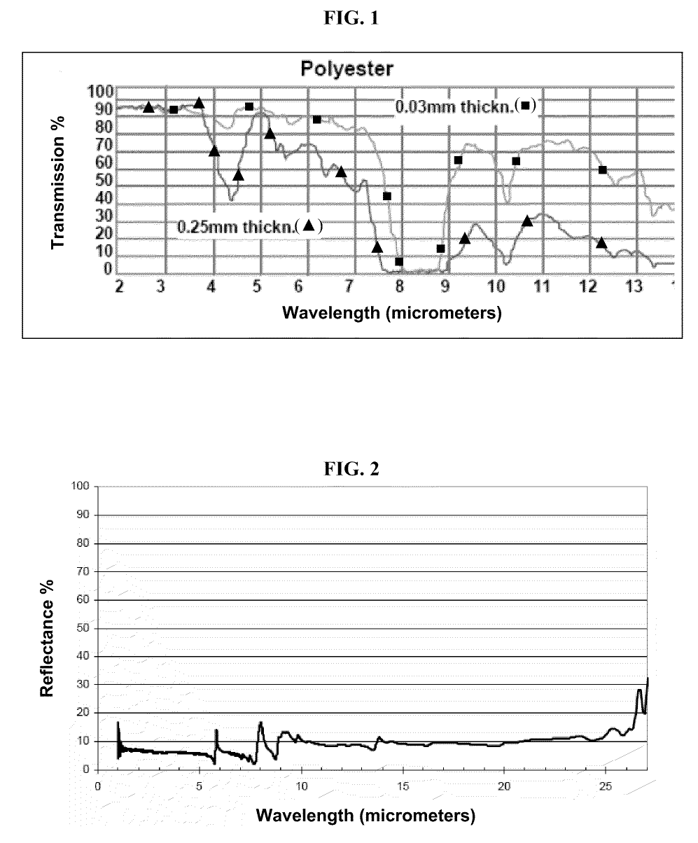

The basic feasibility of infrared temperature measurement was confirmed through testing to determine the conditions under which a target object, preferably a test strip, is opaque to infrared (if a target object is transparent to infrared, objects behind the target could introduce error to the temperature estimation). Infrared transmission was assessed with respect to two different test strips having a thickness of 0.03 mm and 0.25 mm, respectively, each comprising a polyester base material. It was found that when infrared radiation having a wavelength in the range of about 8 microns to about 14 microns is used, the base material of both strips does not transmit infrared to a significant degree (FIG. 1). The thickness of a typical glucose strip is greater than 0.5 mm, and therefore the rate of infrared transmission will be even smaller than that observed with respect to the experimental test strips.

The test strip material was also tested for infrared reflectance. A target surface for infrared temperature measurement should have low infrared reflectance; a material with high infrared reflectance can reflect infrared radiation originating from nearby objects, which leads to erroneous temperature readings. As depicted in FIG. 2, it was determined that the infrared (1 μm to 25 μm) reflectance of polyester test strip material is low at preferred wavelengths (e.g., about 8 μm to about 14 μm).

In some embodiments of the present invention, the infrared sensor is entirely disposed within the housing of the analyte measurement system and assesses a temperature associated with the test strip on a portion of the strip that is inserted into the aperture of the analyte measurement component. Preferably, the assessment of the temperature associated with a portion of the test strip that is inserted into the aperture occurs within about 5 seconds or less, about 4 seconds or less, about 3 seconds or less, about 2 seconds or less, about 1 second or less, or about 0.5 seconds or less from the time of insertion of the test strip. Because the thermo mass of a test strip is low, the test strip will tend to equilibrate to the temperature inside of the housing within a short period of time; however, the infrared sensor of the present invention is capable of rapidly measuring the temperature of the inserted portion of the test strip (target temperatures can be read within milliseconds), and the temperature of the test strip shortly after insertion into the aperture of the analyte measurement component represents a good indicator of the ambient temperature and therefore of the temperature at which the biological sample interacts with reaction zone of the test strip. In accordance with such embodiments, the infrared sensor is preferably positioned within the housing such that the distance between the infrared sensor and the portion of the test strip that is inserted into the aperture of the analyte measurement component is small, for example, less than about 3 mm, less than about 2 mm, less than 1 mm, less than about 0.5 mm, or less than about 0.1 mm.

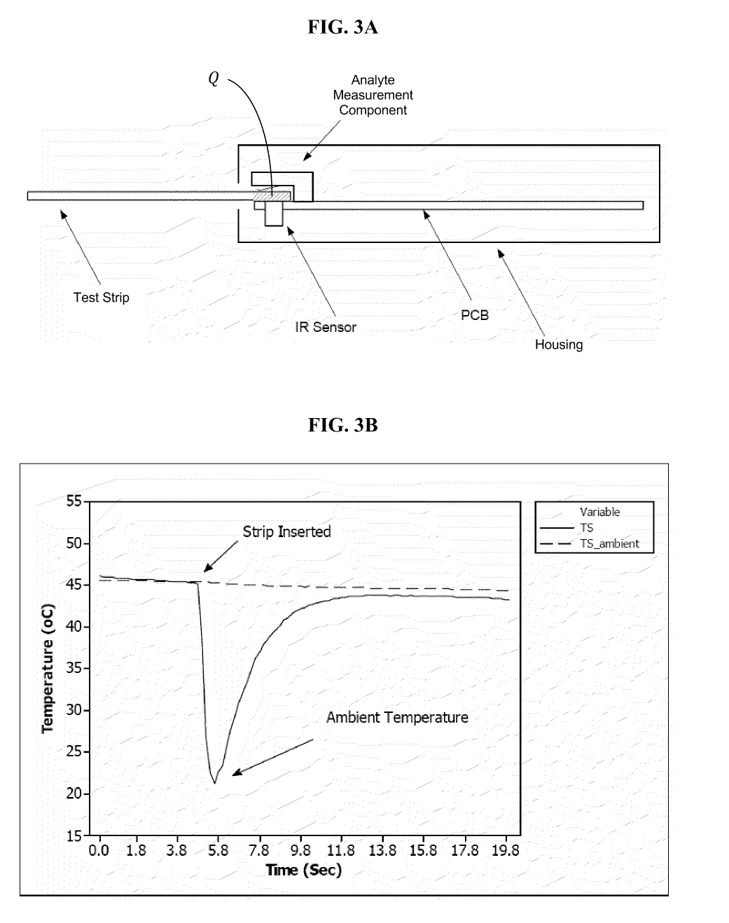

FIG. 3A depicts an exemplary embodiment featuring an infrared sensor disposed within the housing of an analyte measurement system that can measure a portion Q of the test strip (shaded with diagonal lines) that is inserted into the aperture of the analyte measurement component. FIG. 3B provides the results of infrared temperature measurement of a portion of a test strip that was inserted into an analyte measurement system. FIG. 3B shows that although the temperature of the infrared sensor (TS_ambient) was elevated relative to the ambient environment, the sensor was still able to provide accurate temperature measurements of the inserted portion of the test strip. The temperature measurements made by the infrared sensor demonstrated that, following insertion of the strip into the aperture of the analyte measurement component, the temperature of the inserted portion of the strip (TS) initially matched that of the ambient environment outside of the housing (see, e.g., at time≈5.8 seconds), but over time equilibrated to the temperature inside the housing and of the infrared sensor.

Under certain circumstances, even where a temperature measurement of an inserted portion of a test strip is acquired soon after insertion, such measurement may not always provide an accurate representation of the ambient temperature outside of the biosensing instrument. For example, prolonged handling of the test strip by the user during the insertion process may elevate the temperature of the strip beyond that of the ambient environment. Because of this potential limitation, it may be desirable to obtain temperature measurements associated with a portion of the test strip that is not inserted into the aperture of the analyte measurement component. The low thermo mass of the test strip will cause the portion of the strip that is outside of the biosensing instrument to equilibrate to the ambient temperature soon after insertion. Accordingly, some embodiments may include the measurement of a portion of the strip that is not inserted into the analyte measurement component.

In certain embodiments, the present system may further comprise a light guide for directing infrared radiation from a location associated with the test strip to the infrared sensor. The light guide also allows the infrared sensor to focus on the location associated with the test strip. The light guide may be any component that functions as an optical waveguide with respect to infrared radiation that is transmitted from the test strip, the sample on the test strip, or another location associated with the test strip, such that the infrared radiation is directed to the infrared sensor. Planar, tube/pipe, strip, slab, cone, rectangular, pyramidal, and fiber waveguides are exemplary light guides, the characteristics of which may be readily appreciated by those skilled in the art. As used herein, a light guide may also refer to a reflector that reflects infrared radiation originating from a location associated with the test strip to the infrared sensor, and/or focuses the infrared radiation emitted from the location associated with the test strip. Reflectors may be planar, substantially planar, or parabolic. Infrared reflectors are widely recognized among those skilled in the art and are available from various commercial sources. Regardless of the type of light guide that is used, the light guide and the infrared sensor should be substantially isothermic. In a preferred embodiment, the light guide is a light pipe. Infrared light pipes are known among those skilled in the art, and preferably have low infrared emissivity and high infrared reflectance. In addition, there should be sufficient thermal conductivity between the infrared sensor and the light pipe, such that as the sensor begins to heat up during use, the light pipe substantially acclimates to the temperature of the sensor. To this end, to the extent that any material is used to form a connection between the light guide and the infrared sensor, such material should be thermally conductive. Exemplary infrared light pipes include internally gold-coated pipes, which can provide infrared reflectance exceeding about 98%. Light pipes with infrared-reflecting coatings may be straight, curved, or jointed, and preferably feature polished bores. The diameter of any given portion of the light pipe may be less than 1 mm, between about 0.5 mm to about 10 mm, between about 0.5 mm to about 5 mm, or any other suitable diameter. Infrared light pipes are commercially available from a number of sources, such as Epner Technology, Inc. (Greenpoint, N.Y.).

In one embodiment, the infrared sensor is entirely disposed within the housing of the analyte measurement system, and the light guide extends from the sensor lens to an opening in the housing that is located proximate to the analyte measurement component, and by extension proximate to a strip that is inserted in the aperture of the analyte measurement system. The opening permits infrared radiation from a location associated with the test strip to enter the light guide, which directs the radiation to the infrared sensor lens. The opening may be protected by a cover or screen that is infrared-transparent but blocks other unwanted light and protects the light guide, infrared sensor, and other components internal to the housing from dust and other contaminants from the ambient environment. The cover or screen may be a conventional plastic infrared port cover, such as are commonly used on laptop computers, PDAs, and cellular telephones. When in place, the outer surface of the cover or screen may be contiguous or flush with the outer surface of the housing.

The infrared sensor and any components used in directing infrared radiation to the sensor are preferably selected such that the sensor field of view is substantially filled with the target (e.g., with the portion of the strip from which temperature measurement is acquired) and so that the sensor is capable of obtaining temperature readings from a distance relative to the target. If the target does not occupy substantially all of the sensor field of view, infrared radiation from sources other than the target could be detected by the sensor, which could affect the ability of the infrared sensor to accurately determine the temperature associated with the test strip. Accordingly, the opening in the housing that is located proximate to the analyte measurement component into which infrared radiation enters may be sized so that the sensor field of view is substantially filled with the target. The infrared lens of the sensor may be selected to focus on a circumscribed portion of the test strip. One or more infrared reflectors may be included in order to direct the infrared radiation emitted from the target and/or focus the infrared radiation emitted from the target. When present, an infrared reflector is preferably mounted substantially within the housing and serves to reflect infrared radiation that is emitted from the target and received through an opening in the housing; the reflection of infrared radiation directs, focuses, or both directs and focuses the infrared radiation onto the infrared sensor. As discussed previously, any other type of light guide may be included in order to direct the infrared radiation from a location associated with the test strip to said infrared sensor. Preferably, any such component is included in a manner that allows it to be substantially isothermic with the infrared sensor.

The infrared sensor interfaces with a processor that is disposed within the housing and uses temperature data from the sensor to modulate the analyte measurement data acquired by the analyte measurement component. The processor that receives the analyte measurement data may be the same processor that receives temperature data from the infrared sensor. Alternatively, the processor that modulates the analyte measurement data using the temperature data may be a central processing unit that receives the temperature data and the analyte measurement data, respectively, from other processor components. For example, infrared sensor interface electronics may receive temperature data directly from the infrared sensor and deliver such data to a central processing unit.

Infrared sensors themselves are sensitive to temperature changes. In particular, the response of the infrared “thermopile”, the element that performs the actual infrared measurement, is sensitive to temperature. Therefore, the system must take into account the temperature of the infrared sensor in order to make an accurate measurement of the target temperature. Commercially available sensors typically have an embedded thermistor; with respect to such sensors, the temperature of the ambient environment around the sensor is measured and then the infrared sensor response is corrected based on the temperature of the sensor. The sensor thermopile provides a voltage (V_{Target}) that is proportional to the difference of the target temperature (T_{Target}) to the nth power and sensor ambient temperature (T_{Ambient}) to the nth power:

V_{Target}=K×ε×(T_{Target}ⁿ−T_{Ambient}ⁿ)

wherein V_{Target} is the voltage produced by the infrared sensor when it is reading the infrared emission from the target, K is a constant that depends on the sensor and infrared optic efficiency, ε is the emissivity of the target, T_{Target} is the temperature of the target, T_{Ambient} is the ambient temperature around the infrared sensor, and n is preferably 4.

The measured voltage is proportional to the infrared radiation from the target and this is why the exponent n is preferably 4. In practice, n and K are determined during a standard sensor calibration process and c is defined based on the target material. Each of these coefficients may be known in advance and stored in the device memory; under such circumstances, T_{Ambient} is measured by the thermistor component of the infrared sensor, and the target temperature is calculated from the following equation:

$${T}_{Target} = \sqrt[n]{\frac{{V}_{Target}}{K \times ɛ} + {T}_{Ambient}^{n}}$$

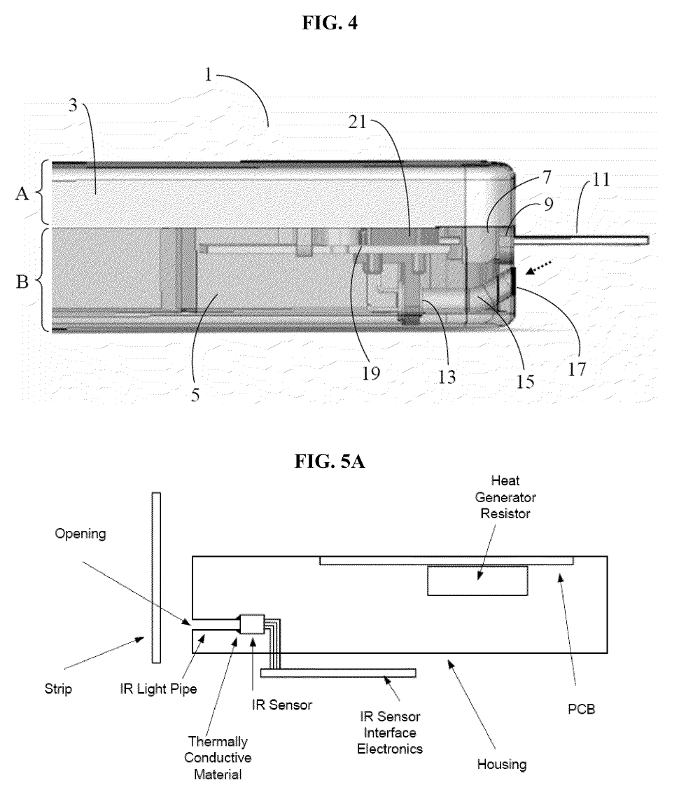

When this algorithm is used the response of the infrared sensor is relatively insensitive to changes in temperature, as shown in Example 1, infra (FIGS. 4B and 4C).

The product literature for an infrared sensor that is commercially available from Melexis Microelectronic Systems (Concord, N.H.; cat. no. MLX90614) includes a chart depicting the achieved accuracy over different target (y-axis) and ambient (x-axis) temperature ranges for that sensor. The product literature is hereby incorporated herein by reference in its entirety. Catalogue number MLX90614 is an appropriately sized sensor that is said to have a temperature accuracy of ±0.5° C. over a wide temperature range. The range of 0° C.-60° C. corresponds to the temperatures at which it may be expected that a biosensing instrument would be operated, while the infrared sensor temperature could vary between 0° C. to 50° C.; the error of ±0.5° C. within the convergence of these ranges indicates that this device is well suited for ambient temperature measurements in the context of analyte measurement.

As indicated previously, numerous commercially available infrared sensors are available for use in connection with the present systems. Some of the commercially available infrared temperature sensors are integrated, featuring the sensor component, additional thermistor, and analog and digital interface circuits. Examples of such devices are catalogue numbers MLX90614 and MLX90615 from Melexis Microelectronic Systems (Concord, N.H.). These devices are self-contained and only require power and serial lines for operation. MLX90615 has a much smaller form factor and is preferred for use with compact systems. Other commercially available sensors have only analogue circuitry and necessitate the use of an external A/D converter for further data processing. Examples of such devices include item number A2TPMI 23 S from PerkinElmer Optoelectronics (Fremont, Calif.), and the HIS module from Heimann Sensor GmbH (Dresden, Germany). Still other commercially available sensors feature the infrared sensor and thermistor only, such that external processing electronics are required to measure temperature. The benefit of these devices is that they are very small. Examples include ZTP 135 from General Electric Sensing & Inspection Technologies (Billerica, Mass.), ST60R and ST60 Micro from Dexter Research, Inc. (Dexter, Mich.), HMS Z11 F5.5 from Heimann Sensor GmbH (Dresden, Germany), and TPS 23 S from PerkinElmer Optoelectronics (Fremont, Calif.).

The modulation of the analyte measurement data may include compensating for the assessed temperature associated with the test strip during a measurement of an analyte on the test strip. In other embodiments, the present methods may include modulating data acquired during a measurement of an analyte on the test strip to compensate for the assessed temperature associated with the test strip. The modulated analyte measurement data may then be conveyed to the user. The analyte measurement system may include a display for displaying the modulated analyte measurement data, and may also or alternatively include audio components so that the modulated analyte measurement data may be conveyed using sound. For example, “talking” glucose meters include speaker components that allow a visually impaired user to hear the results of a blood glucose analysis. The user may consider the modulated data in order to decide whether a medication regimen, doctor visit, or other medical intervention is necessary.

FIG. 4 depicts a partially transparent side view of an exemplary analyte measurement system 1 as it would appear if placed in a horizontal resting position on a flat surface, e.g., a tabletop. The housing 3 that substantially defines internal space 5 is shown as opaque in upper portion A, while lower portion B allows the internal components of the system 1 to be viewed as though the housing were cut away. An analyte measurement component 7 is disposed within the housing 3 and features an aperture 9 for receiving a test strip 11. An infrared sensor 13 is also disposed within the housing 3 for assessing a temperature associated with the test strip 11. An infrared light pipe 15 extends from the sensor 13 to an opening in housing 3 at a location proximate the test strip 11. The opening includes an infrared port cover 17 that allows infrared radiation (arrow) to travel from a location associated with the test strip 11 while preventing dust or other contaminants from the ambient environment from entering the light pipe 15. A circuit board 19 accommodates the microprocessor 21 and allows both the infrared sensor 13 and the analyte measurement component 7 to interface with the microprocessor 21. The infrared sensor 13 interfaces with a microprocessor 21 to provide temperature data regarding the location associated with the test strip thereto. The analyte measurement component 7 also interfaces with the microprocessor 21 to provide analyte measurement data, which is modulated by the microprocessor 21 in view of the received temperature data.

### EXAMPLES

### Example 1

### System with Infrared Sensor and Basic Light Guide

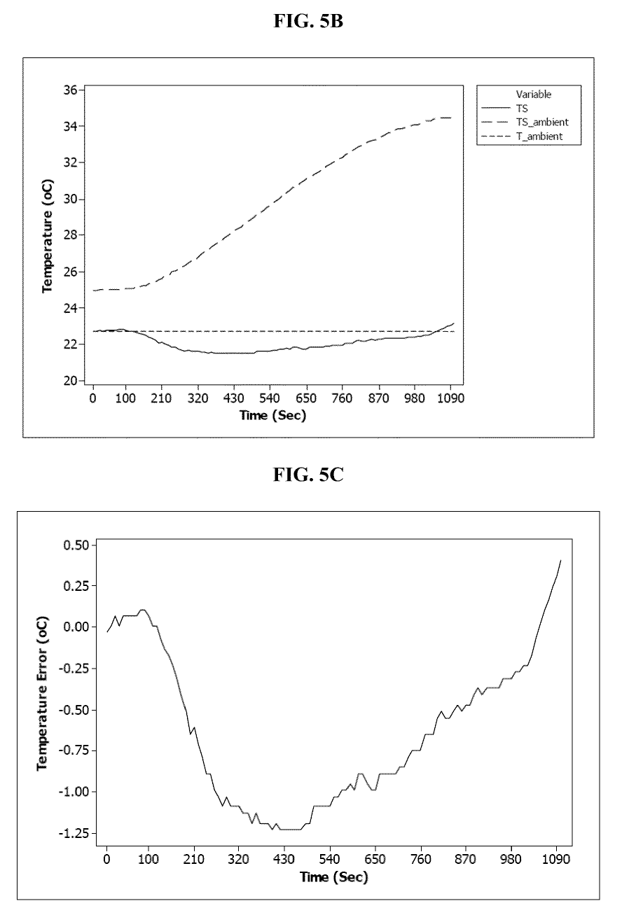

To demonstrate the feasibility of the concept of the use of an internal infrared sensor, a straight infrared light pipe with an inner diameter of 3.8 mm and length of 10 mm was attached to an MLX90615 infrared sensor (Melexis Microelectronic Systems, Concord, N.H.). Thermally conductive heat sink compound was used to attach the light pipe and the sensor. The assembly was mounted inside a housing that also featured a heat-generating resistor. The resistor was attached to a power supply to generate heat within the housing. FIG. 5A depicts the resulting arrangement of components.

The infrared sensor was used to measure the temperature of a standard glucose strip positioned outside of the device. The results are summarized in FIG. 5B. The temperature of the infrared sensor itself (“TS_ambient”) increased significantly over time without introducing significant error to the measurement of the target temperature (“TS”), which ideally represents an approximation of the ambient temperature of the outside environment surrounding the device (“T_ambient”). FIG. 5C shows the error in the temperature measurement obtained by the infrared sensor. The error is attributable to the rapidly-changing temperature of the infrared sensor due to conditions within the device housing. The margin of error was not more than 1.2° C., demonstrating that frequent changes in temperature within the device will not interfere with the ability of the infrared sensor to measure the temperature of a target outside of the device.

The preceding test was performed using a basic prototype, and no particular optic alignment was performed in order to optimize the performance of the system. In addition, to expedite the testing, the power dissipation within the device was increased significantly so that a rapid change in the temperature would result; although rapid changes in the infrared sensor temperature or thermal shocks could degrade the accuracy of the infrared temperature measurement, under conditions of actual usage, the internal device temperature would not fluctuate as rapidly. Thus, an optimized system is expected to have a lower margin of error than the device used for purposes of the present experiment.

The disclosures of each patent, patent application, and publication cited or described in this document are hereby incorporated herein by reference, in their entirety.

As employed above and throughout the disclosure, the following terms and abbreviations, unless otherwise indicated, shall be understood to have the following meanings. In the present disclosure the singular forms “a,” “an,” and “the” include the plural reference, and reference to a particular numerical value includes at least that particular value, unless the context clearly indicates otherwise. Thus, for example, a reference to “a processor” is a reference to one or more of such processors and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive, divisible, and combinable. For example, when a range of “1 to 5” is recited, the recited range should be construed as including ranges “1 to 4”, “1 to 3”, “1 to 2”, “1 to 2 and 4 to 5”, “1 to 3 and 5”, and the like.

What is claimed:

1. A method comprising using an infrared sensor to assess temperature associated with a test strip that is inserted into an analyte measurement system,
wherein said system comprises
a housing;
an analyte measurement component disposed within said housing, or proximate said housing, and having an aperture for receiving said test strip, wherein said analyte measurement component measures an analyte on said test strip, thereby providing analyte measurement data;
said infrared sensor disposed at least partially within said housing; and
a processor disposed within said housing that uses temperature data from said infrared sensor to modulate said analyte measurement data.

2. The method according to claim 1 wherein said infrared sensor is disposed substantially within said housing.

3. The method according to claim 1 wherein said test strip is an electrochemical test strip.

4. The method according to claim 1 wherein said system further comprises a light guide for directing infrared radiation from a location associated with the test strip to said infrared sensor.

5. The method according to claim 4 wherein said light guide and said infrared sensor are substantially isothermic.

6. The method according to claim 4 wherein said light guide comprises a light pipe.

7. The method according to claim 1 wherein said infrared sensor uses infrared radiation having a wavelength of about 8 μm to about 14 μm to assess said temperature associated with said test strip.

8. The method according to claim 1 wherein said test strip has a length l and wherein said temperature is assessed on a portion of said test strip that is located at a distance of no more than about ⅓l from the end of the test strip that is inserted into the aperture of said analyte measurement component.

9. The method according to claim 1 wherein said infrared sensor assesses said temperature on a portion of the strip that is inserted into the aperture of the analyte measurement component.

10. The method according to claim 9 wherein said temperature is assessed within about 5 seconds or less following insertion of the test strip into the aperture of the analyte measurement component.

11. The method according to claim 1 wherein said test strip has a length l and wherein said temperature is assessed on a portion of said test strip that is located at a distance that is greater than about ⅓l from the end of the test strip that is inserted into the aperture of said analyte measurement component.

12. The method according to claim 1 wherein said test strip has a length l and wherein said temperature is assessed on a portion of said test strip that is located at a distance that is greater than about ⅔l from the end of the test strip that is inserted into the aperture of said analyte measurement component.

13. The method according to claim 1 further comprising compensating for said assessed temperature associated with said test strip during a measurement of an analyte on said test strip.

14. The method according to claim 1 further comprising modulating data acquired during a measurement of an analyte on said test strip to compensate for said assessed temperature associated with said test strip.

15. The method according to claim 1 wherein said analyte measurement system is a blood glucose meter.

16. The method according to claim 1 wherein said temperature is assessed on more than one location on said test strip.

17. The method according to claim 1 wherein said temperature associated with said test strip is assessed more than one time.

18. The method according to claim 17 wherein said temperature is assessed on more than one location on said test strip.

19. A system comprising:
a housing;
an analyte measurement component disposed within said housing, or proximate said housing, and having an aperture for receiving a test strip, wherein said analyte measurement component measures an analyte on said test strip, thereby providing analyte measurement data;
an infrared sensor disposed at least partially within said housing; and
a processor disposed within said housing that uses temperature data from said infrared sensor to modulate said analyte measurement data.

20. The system according to claim 19 wherein said infrared sensor is disposed substantially within said housing.

21. The system according to claim 19 wherein both of said analyte measurement component and said infrared sensor are in electronic communication with said processor.

22. The system according to claim 19 wherein said system further a light guide for directing infrared radiation from a location associated with the test strip to said infrared sensor.

23. The system according to claim 22 wherein said light guide and said infrared sensor are substantially isothermic.

24. The system according to claim 22 wherein said light guide comprises a light pipe.

25. The system according to claim 19 wherein said infrared sensor uses infrared radiation having a wavelength of about 8 μm to about 14 μm to assess said temperature associated with said test strip.

26. The system according to claim 19 wherein said test strip has a length l and wherein said infrared sensor assesses temperature on a portion of said test strip that is located at a distance of no more than about ⅓l from an end of the test strip that is inserted into the aperture of said analyte measurement component.

27. The system according to claim 19 wherein said infrared sensor assesses said temperature on a portion of the strip that is inserted into the aperture of the analyte measurement component.

28. The system according to claim 19 wherein said test strip has a length l and wherein said infrared sensor assesses temperature on a portion of said test strip that is located at a distance that is greater than about ⅓l from an end of the test strip that is inserted into the aperture of said analyte measurement component.

29. The system according to claim 19 wherein said test strip has a length l and wherein said infrared sensor assesses temperature on a portion of said test strip that is located at a distance that is greater than about ⅔l from an end of the test strip that is inserted into the aperture of said analyte measurement component.

30. The system according to claim 19 wherein said analyte measurement component measures blood glucose.

31. The system according to claim 19 further comprising a display for displaying said modulated analyte measurement data.
